(19) United States
(12) Patent Application Publication
(10) Pub. No.: US 2025/0281575 A1
(43) Pub. Date: Sep. 11, 2025

(54) METHODS FOR THE TREATMENT OF DIABETES-ASSOCIATED AUTONOMIC NEUROPATHY

(71) Applicant: SONNET BIOTHERAPEUTICS, INC., Princeton, NJ (US)

(72) Inventors: Gael Hédou, Yvonand (CH); Yves Sagot, Beaumont (FR)

(21) Appl. No.: 19/068,788

(22) Filed: Mar. 3, 2025

Related U.S. Application Data
(60) Provisional application No. 63/561,924, filed on Mar. 6, 2024.

(51) Int. Cl. A61K 38/20

(52) U.S. Cl. CPC A61K 38/204

(57) ABSTRACT

Provided herein are novel methods for the treatment of diabetes-associated autonomic neuropathies using IL-6 compositions.

### CROSS-REFERENCE TO RELATED APPLICATIONS

The U.S. Patent Application claims benefit of U.S. Provisional Patent Application No. 63/561,924, filed on Mar. 6, 2024, which is hereby incorporated by reference in its entirety.

### BACKGROUND

Autonomic neuropathies are complex disorders that occur as a result of damage to the nerves that control autonomic body functions. Autonomic neuropathies result in diverse clinical manifestations that affect the cardiovascular, gastrointestinal, urogenital, and sudomotor systems. Autonomic neuropathy can cause, for example, difficulties in adapting to changes in posture or activity level, erectile dysfunction, incontinence, gastrointestinal disturbances, and an increased risk of cardiovascular morbidity.

A major cause of autonomic neuropathy is diabetes mellitus. Neuropathy (peripheral and autonomic neuropathy) are known to occur in 30-40% of patients. Diabetic autonomic neuropathies are a heterogeneous and progressive disease entity and commonly complicate both type 1 and type 2 diabetes mellitus. Although the etiology is not entirely understood, hyperglycemia, insulin deficiency, metabolic derangements and potentially autoimmune mechanisms are thought to play an important role. A subgroup of diabetic autonomic neuropathy, cardiovascular autonomic neuropathy (CAN), is one of the most common diabetes-associated complications and is ultimately clinically important because of its correlation with increased mortality. Although silent in the earlier stages, it is a powerful predictor of mortality risk in diabetic patients and is a major challenge for all physicians dealing with people suffering from diabetes. Patients with CAN have a five-fold increased risk of mortality due to a high-risk of cardiac arrhythmias, silent myocardial ischemia and sudden death.

Thus, there is a need for effective therapies for the treatment of diabetes-associated autonomic neuropathy.

### SUMMARY

Provided herein are methods for treating a diabetes-associated autonomic neuropathy in a subject in need thereof, the method comprising to the subject a therapeutically effective dose of exogenous interleukin-6 (IL-6). In some embodiments, the autonomic neuropathy is associated with type 1 diabetes (T1D). In some embodiments, the autonomic neuropathy is associated with type 2 diabetes (T2D).

In some embodiments, the IL-6 reduces a stomach or intestine symptom associated with autonomic neuropathy selected from the following: constipation, diarrhea, early satiety, nausea after eating, bowel movement control, swallowing, swollen abdomen, vomiting of undigested food, or a stomach or intestine symptom that is associated with related autonomic nerve degeneration.

In some embodiments, the IL-6 reduces a heart or lung symptom associated with autonomic neuropathy selected from the following: abnormal heart rate or rhythm, blood pressure drop with position, high blood pressure, or shortness of breath with activity or exercise.

In exemplary embodiments, the IL-6 reduces a bladder symptom associated with autonomic neuropathy selected from the following: difficulty in initiation of urination, feeling of incomplete bladder emptying, or uncontrolled urination.

In some embodiments, the IL-6 reduces one or more of the following symptoms associated with autonomic neuropathy: excessive or reduced sweating, heat intolerance associated with activity or exercise, erectile dysfunction, vaginal dryness, female orgasm difficulty, reduced pupil size, or unexpected weight loss.

In some embodiments, the IL-6 restores a physiological balance of parasympathetic/sympathetic tone in the subject. In exemplary embodiments, the IL-6 reduces tachycardia or bradycardia in the subject.

In some embodiments, the IL-6 is administered to the subject at a dose of 0.01 μg/kg to 10 μg/kg. In exemplary embodiments, the IL-6 is administered to the subject at a dose of 0.1 μg/kg to 1 μg/kg.

### DETAILED DESCRIPTION

Provided herein are methods for the treatment of diabetes-associated autonomic neuropathy using a composition that includes IL-6 (e.g., exogenous human IL-6). Aspects of the subject methods are described in detail below.

### I. Interleukin-6 Composition

In the subject methods provided herein, an IL-6 composition is administered to a patient having an autonomic neuropathy.

In some embodiments, the IL-6 treatment includes a recombinant IL-6, or a biologically active IL-6 variant or biologically active fragment thereof. In particular embodiments, the IL-6 is a human IL-6, or a biologically active human IL-6 variant or biologically active fragment thereof.

As used herein, “IL-6,” “IL6,” “BSF2,” “HGF,” “HSF,” “IFNB2,” “BSF-2,” “B cell stimulatory factor 2,” “CDF,” “IFN-beta-2,” and “interleukin 6” (Genbank Accession numbers: NM_000600, NM_001318095, NP_000591 and NP_001305024 (human IL-6); and NM_031168, NM_001314054, NP_001300983, and NP_112445 (mouse IL-6)) all refer to an interleukin that acts as both a pro-inflammatory cytokine and an anti-inflammatory myokine. IL-6 signals though a cell-surface type I cytokine receptor complex that consists of the IL-6Rα chain (also referred to as “gp 80”) and gp 130 (also referred to as “CD130”).

Interleukin-6 (IL-6) is a multifunctional cytokine produced and secreted by several different cell types. IL-6 is a 20 to 26 kDa glycoprotein having 185 amino acids that has been cloned previously (May et al, (1986); Zilberstein et al, (1986); Hirano et al, (1986)). IL-6 is secreted by a number of different tissues including the liver, spleen, and bone marrow and by a variety of cell types including monocytes, fibroblasts, endothelial, B- and T-cells. IL-6 is activated at the transcriptional level by a variety of signals including viruses, double stranded RNA, bacteria and bacterial lipopolysaccharides, and inflammatory cytokines such as IL-1 and TNF.

The biological activities of IL-6 are mediated by a membrane receptor system comprising two different proteins: IL-6 receptor (gp80) and gp130. Soluble forms of IL-6R gp80 (sIL-6R), corresponding to the extracellular domain of gp80, are natural products of the human body found as glycoproteins in blood and in urine (Novick et al, 1990, 1992). An exceptional property of sIL-6R molecules is that they bind IL-6 and the IL-6/IL-R complex acts as an agonist of gp130 on many cell types including human cells. Even without the intracytoplasmic domain of gp80, sIL-6R is still capable of triggering the dimerization of gp130 in response to IL-6, which in turn mediates the subsequent IL-6-specific signal transduction and biological effects (Murakami et al, 1993). sIL-6R has two types of interaction with gp130 both of which are essential for the IL-6 specific biological activities (Halimi et al., 1995), and the active IL-6 receptor complex was proposed to be a hexameric structure formed by two gp130 chains, two IL-6R and two IL-6 ligands (Ward et al., 1994; Paonessa et al, 1995).

In some embodiments, the IL-6 used in the subject methods is a biologically active variant of IL-6. Biologically active variant IL-6s include one or more of the amino acid residues of the naturally occurring components of IL-6 are replaced by different amino acid residues, or are deleted, or one or more amino acid residues are added to the original sequence of an IL-6, without changing considerably the activity of the resulting products as compared with the original IL-6. These variant IL-6s are prepared by known synthesis and/or by site-directed mutagenesis techniques, or any other known technique suitable therefore.

In some embodiments, the IL-6 used in the subject methods exhibit substantially similar, or even better, activity to wild-type IL-6 (e.g., human IL-6). In some embodiments, biological activity of IL-6 is determined as the capability of binding to the gp80 portion of the IL-6 receptor and/or the capability of inducing hepatocyte proliferation, it can be considered to have substantially similar activity to IL-6. Thus, it can be determined whether any given IL-6 variant has at least substantially the same activity as IL-6 by means of routine experimentation comprising subjecting hepatocytes to such mutein, and to determine whether or not it induces hepatocyte proliferation e.g. by measuring BrdU or labelled methionine uptake or just by counting non treated control cells and cells treated with wild-type IL-6. An Enzyme Linked ImmunoSorbent Assay (ELISA) type assay for measuring the binding of IL-6R/IL-6 chimera to gp130 has been described in detail, for example, WO 99/02552. In particular embodiments, an IL-6 is considered to have substantially similar biological activity to IL-6 if it has substantial binding activity to its binding region of gp80.

In a preferred embodiment, the variant IL-6 has at least 40% identity or homology with the sequence of mature wild-type human IL-6. In some embodiments, the variant IL-6 has at least 50%, at least 60%, at least 70%, at least 80%, or at least 90% identity or homology thereto. In some embodiments, the IL-6 used in the subject methods includes at least 95%, 96%, 97%, 98%, 99% or 100% identity to wild-type human IL-6.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus, for instance, programs available in the Wisconsin Sequence Analysis Package, version 9.1 (Devereux J et al. 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990; Pearson 1988). Sequence identity between two similar sequences can also be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol.48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm. When using any of the aforementioned algorithms, the default parameters (for Window length, gap penalty, etc.) are used. In one embodiment, sequence identity is done using the BLAST algorithm, using default parameters.

In some embodiments, the IL-6 used in the subject methods described herein includes one or more “conservative” substitutions. Conservative amino acid substitutions of IL-6 may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Examples of production of amino acid substitutions in proteins which can be used for obtaining variants of IL-6 polypeptides, for use herein include any known method steps, such as presented in U.S. Pat. Nos. 4,588,585; 4,737,462; 5,116,943; 4,965,195; 4,879,111; 5,017,691; and 4,904,584. Specific IL-6 variants, which are useful herein have been described, for example, in WO1994003492, U.S. Pat. Nos. 5,681,723, 5,789,552, which is incorporated by reference in relevant parts relating to IL-6 variants.

In some embodiments, the IL-6 used in the subject methods is fused to a soluble IL-6 receptor. Chimeric IL-6R/IL-6 molecules linking the soluble IL-6 receptor and IL-6 together have been developed (Chebath et al. Eur Cytokine Netw. 8(4):359-65 (1997)). The chimeric IL-6R/IL-6 molecules were generated by fusing the entire coding regions of the cDNAs encoding the soluble IL-6 receptor (sIL-6R) and IL-6. Recombinant IL-6R/IL-6 was produced in CHO cells (Chebath et al, Eur Cytokine Netw. 1997, WO99/02552). The IL-6R/IL-6 binds with a higher efficiency to the gp130 chain in vitro than does the mixture of IL-6 with sIL-6R (Kollet et al, Blood. 1999 Aug. 1; 94(3):923-31).

The IL-6 and IL-6R/IL-6 described herein may be produced in any adequate eukaryotic or prokaryotic cell type, like yeast cells, insect cells, bacteria, and the like. In one embodiment, IL-6 is produced in mammalian cells, such as in genetically engineered CHO cells as described in WO 99/02552.

In some embodiments, the IL-6 used in the subject methods is not glycosylated. Advantageously, the molecule can then be produced in bacterial cells, which are not capable of synthesizing glycosyl residues, but usually have a high yield of produced recombinant protein. The production of non-glycosylated IL-6 has been described in detail in EP504751B1, which is incorporated by reference in pertinent parts for teaching methods for making non-glycosylated IL-6.

Formulations of the IL-6 used in accordance with the methods provided herein are prepared for storage by mixing an IL-6 having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA or DTPA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, polyethylene glycol (PEG) and/or polysorbate.

The formulation herein may also contain more than one active compound as necessary for the particular indication being treated, preferably those with complementary activities that do not adversely affect each other.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the IL-6 composition provided herein, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

When encapsulated the subject IL-6 compositions provided herein remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

In some embodiments, the IL-6 for use in the subject methods is in a formulation that includes: about 2 μg/ml to about 2 mg/ml IL-6; about 5 to about 15 mM histidine; about 5 to about 15 mg/ml glycine; about 40 to about 60 mg/mL trehalose; about 0.1 to about 0.3 mg/mL polysorbate 20; and about 1 to about 1000 μM DTPA, wherein the formulation has a pH of about 6.0 to about 8.0. In some embodiments, the IL-6 is human IL-6 or recombinant human IL-6 (rh IL-6).

In some embodiments, the formulation comprises 2 μg/mL, 5 μg/mL IL-6, 10 μg/mL, 15 μg/mL IL-6, 20 μg/mL IL-6, 25 μg/mL IL-6, 30 μg/mL IL-6, 35 μg/mL IL-6, 40 μg/mL IL-6, 45 μg/mL IL-6, 50 μg/mL IL-6, 55 μg/mL IL-6, 60 μg/mL IL-6, 65 μg/mL IL-6, 70 μg/mL IL-6, 75 μg/mL IL-6, 80 μg/mL IL-6, 85 μg/mL IL-6, 90 μg/mL IL-6, 95 μg/mL IL-6, 100 μg/mL IL-6, 105 μg/mL IL-6, 110 μg/mL IL-6, 115 μg/mL IL-6, 120 μg/mL IL-6, 125 μg/mL IL-6, 130 μg/mL IL-6, 135 μg/mL IL-6, 140 μg/mL IL-6, 145 μg/mL IL-6, 150 μg/mL IL-6, 155 μg/mL IL-6, 160 μg/mL IL-6, 165 μg/mL IL-6, 170 μg/mL IL-6, 175 μg/mL IL-6, 180 μg/mL IL-6, 185 μg/mL IL-6, 190 μg/mL IL-6, 195 μg/mL IL-6, 200 μg/mL IL-6, 205 μg/mL IL-6, 210 μg/mL IL-6, 215 μg/mL IL-6, 220 μg/mL IL-6, 225 μg/mL IL-6, 230 μg/mL IL-6, 235 μg/mL IL-6, or 240 μg/mL IL-6. In some embodiments, the formulation comprises 80 μg/mL IL-6. In some embodiments, the formulation comprises 120 μg/mL IL-6. In some embodiments, the formulation comprises 240 μg/mL IL-6.

In some embodiments, the formulation comprises about 10 mM histidine. In some embodiments, the formulation comprises about 10 mg/ml glycine. In some embodiments, the formulation comprises about 50 mg/mL trehalose. In some embodiments, the formulation comprises about 0.2 mg/ml polysorbate 20. In some embodiments, the formulation comprises about 50 μM DTPA. In certain embodiments, the formulation has a pH of about 7.0.

In some embodiments, the IL-6 formulation, comprises: 40 to about 240 μg/mL human IL-6; 10 mM histidine; 10 mg/ml glycine; 50 mg/mL trehalose; 0.2 mg/mL polysorbate 20; and 50 μM DTPA, wherein the formulation has a pH of about 7.0.

In some embodiments, the formulation has a post peak of less than about 3% after 14 days at −80° C. measured by RP-HPLC. In some embodiments, the formulation has a post peak of less than about 3% after 14 days at 2-8° C. measured by RP-HPLC.

### II. Treatment Modalities

The IL-6 compositions provided herein are administered for the treatment of a diabetes-associated (Type 1 or Type 2) autonomic neuropathy in a subject in need thereof. Autonomic neuropathies represent a group of disorders that preferentially target autonomic nerve fibers (i.e., nerves that control automatic involuntary body functions) and can be classified as either acute/subacute or chronic in onset. Acute-onset autonomic neuropathies manifest with such conditions as paraneoplastic syndromes, Guillain-Barre syndrome, Sjögren syndrome, infection, or toxins/chemotherapy. When the presentation is acute, immune-mediated, and without a secondary cause, autoimmune autonomic ganglionopathy is likely. Of the chronic-onset forms, diabetes is the most widespread and disabling, with autonomic impairment portending increased mortality and cardiac wall remodeling risk.

In the methods provided herein, therapy is used to provide a positive therapeutic response with respect to a disease or condition. By “positive therapeutic response” is intended an improvement in one or more symptoms associated with autonomic neuropathy.

Treatment includes a “therapeutically effective amount” of the IL-6 medicaments used. A “therapeutically effective amount” refers to an amount effective, at dosages and for periods of time necessary, to achieve a desired therapeutic result (e.g., a positive therapeutic response as discussed herein). As provided herein, a therapeutically effective amount refers to an amount effective of IL-6, at dosages and for periods of time necessary, to achieve a desired therapeutic benefit.

In some embodiments, the IL-6 reduces a stomach or intestine symptom associated with the diabetes-associated autonomic neuropathy selected from the following: constipation, diarrhea, early satiety, nausea after eating, bowel movement control, swallowing, swollen abdomen, vomiting of undigested food, or a stomach or intestine symptom associated with related autonomic nerve degeneration. Assessment of stomach or intestine symptoms can be carried out using any technique known in the art.

In some embodiments, the IL-6 reduces a heart or lung symptom associated with the diabetes-associated autonomic neuropathy selected from the following: abnormal heart rate or rhythm, blood pressure drop with position, high blood pressure, or shortness of breath with activity or exercise. Assessment of heart and lung symptoms can be carried out using any technique known in the art.

In exemplary embodiments, the IL-6 reduces a bladder symptom associated with the diabetes-associated autonomic neuropathy selected from the following: difficulty in initiation of urination, feeling of incomplete bladder emptying, or uncontrolled urination. Assessment of bladder symptoms can be carried out using any technique known in the art.

In some embodiments, the IL-6 reduces one or more of the following symptoms associated with the diabetes-associated autonomic neuropathy: excessive or reduced sweating, heat intolerance associated with activity or exercise, erectile dysfunction, vaginal dryness, female orgasm difficulty, reduced pupil size, or unexpected weight loss.

In some embodiments, the IL-6 is administered for the treatment of cardiac autonomic neuropathy in a subject. In some embodiments, the cardiac autonomic neuropathy is associated with diabetes in the subject.

In some embodiments, the IL-6 restores a physiological balance of parasympathetic/sympathetic tone in the subject. In some embodiments, the IL-6 reduces tachycardia (e.g., sinus, supraventricular, and ventricular), or bradycardia in the subject. In some embodiments, one or more of the following tests are performed to assess improvement in condition: cardiovascular parasympathetic functions using the deep breathing test, cardiovascular sympathetic functions using Valsalva maneuver and tilt test, and sudomotor functions. In some embodiments, improvement in autonomic function is performed using an Ewing battery of cardiovascular testing consisting of the deep breathing test, Valsalva maneuver, and response to standing or the test that, which can be combined with sudomotor testing or with transcranial Doppler, capnography, and skin biopsies.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the IL-6 used in the methods described herein depend on the condition to be treated and may be determined by the persons skilled in the art. In some embodiments, the IL-6 is administered at a dose of from about 0.01 μg/kg—about 10 μg/kg. In some embodiments, the IL-6 is administered at a dose of from about 0.01—about 0.05 μg/kg, about 0.05—about 0.10 μg/kg, about 0.10—about 0.50 μg/kg, about 0.05—about 1.0 μg/kg, about 1—about 2 μg/kg, about 2—about 3 μg/kg, about 3—about 4 μg/kg, about 4—about 5 μg/kg, about 5—about 6 μg/kg, about 6—about 7 μg/kg, about 7—about 8 μg/kg, about 8—about 9 μg/kg, or about 9—about 10 μg/kg. In exemplary embodiments, the dose is from about 0.05 μg/kg—about 3 μg/kg. In some embodiments, the IL-6 is administered at a dose of from about 0.1 μg/kg—about 1 μg/kg.

In some embodiments, the IL-6 used in the methods described herein is administered at a dose of from about 0.75 μg to 750 μg. In some embodiments, the IL-6 is administered at a dose of from about 1 μg—about 5 μg, about 5 μg—about 10 μg, about 10 μg—about 25 μg, about 25 μg—about 50 μg, about 50 μg—about 100 μg, about 100 μg—about 150 μg, about 150 μg—about 200 μg, about 200 μg—about 250 μg, about 250 μg—about 300 μg, about 300 μg—about 350 μg, about 350 μg—about 400 μg, about 400 μg—about 450 μg, about 450 μg—about 500 μg, about 550 μg—about 600 μg, about 600 μg—about 650 μg, about 650 μg—about 700 μg, or about 700 μg—about 750 μg.

In embodiments, the IL-6 is administered daily, weekly, or monthly. In some embodiments, the IL-6 is administered as a weekly dose for a duration of the treatment. In some embodiments, the IL-6 is administered 1, 2, 3, 4, 5, 6, 7, 8, 9, or 10 times or more a week for the duration of the treatment. In some embodiments, the IL-6 is administered 3 times a week. In some embodiments, the IL-6 is administered, weekly, every other day of the week. In some embodiments, the IL-6 is administered as a monthly dose for a duration of the treatment. In some embodiments, the IL-6 is administered 1, 2, 3, 4, 5, 6, 7, 8, 9, 10, 11 or 12 or more month for the duration of the treatment.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

### III. Administrative Modalities

The subject IL-6 compositions provided herein are administered to a subject, in accord with known methods, such as intravenous, parenteral, intramuscular, intraperitoneal, intracerobrospinal, subcutaneous, intra-articular, intrasynovial, intrathecal, oral, topical, intrarectal, or inhalation administration. Intravenous, parenteral, or subcutaneous administration of the IL-6 composition is preferred.

### EXAMPLES

Examples are provided below to illustrate the present invention. These examples are not meant to constrain the present invention to any particular application or theory of operation.

### Example 1: Effects on IL-6 on Type 1 and Type 2 Diabetes-Associated Autonomic Neuropathy

To assess the effects of IL-6 on autonomic neuropathy associated with type 1 (T1D) and type 2 diabetes (T2D), IL-6 will be administered to a T1D and T2D rodent model. In the T1D study, rodents already treated with streptozotocin (STZ) to induce diabetes and autonomic neuropathy (see, e.g., Nakos et al., In Vivo 32(6):1433-1441 (2018)) are administered IL-6 or vehicle. In the T2D study, rodents fed a high fat or Western diet (WD, see O'Brien et al., ILAR J. 54(3):259-272 (2014); Gilbert et al., Exp. Diabetes Res. 1-12 (2011); and Sumiyoshi et al., J. Nutr. 136 (3):582-587 (2006)) and administered STZ to induce diabetes and autonomic neuropathy (see, e.g., Akinlade et al., Int J. Health Sci 14(6):24-30 (2020); and Elshareif et al., Front Physiol. 14:1238120 (2023)) are treated with IL-6 or vehicle. IL-6 can also be co-administered with STZ or vehicle to the rodent.

One or more of the following evaluations will be subsequently performed with each group:
Echocardiography measurements to assess cardiac dysfunction and heart rate variability (HRV), a common indicator in cardiac autonomic neuropathy (see Yadav et al., Diabetes, Metabolic Syndrome Obes. Targets Ther. 10:57-64 (2017), and Elshareif et al., Front Physiol. 14:1238120 (2023))
Mean arterial blood pressure and heart rate measurements including one or more of the following: systolic and diastolic arterial pressure, heart rate and heart rate variability, bradycardia, left ventricular function
Baroreceptor sensitivity (BRS), an indicator of cardiac autonomic neuropathy, for example, by the vasoactive method (see El Gowelli et al., Life Sci 185:15-22 (2017); and Bakkar et al., Endocrinol Metab. 319(5):E835-E851 (2020))
Body weight measurements.
Daily water and food consumption
Blood concentration measurements of glucose, cholesterol and triglycerides, and/or serum concentrations of NPY or Troponin T.
Measurement of the urinary function by monitoring volume production.
Measurement of gastric emptying, number and weight of daily feces
Sympathetic innervation density measurements using specific markers in immunohistochemical analyses in organs under the control of the autonomic nervous system such as, for example, heart, intestine, bladder, right atrium, using tyrosine hydroxylase, choline acetyltransferase markers
Gastrointestinal dysmotility assessment including, for example, gastric emptying and small bowel transit, colonic transit and morphological studies. See, e.g., Domenech et al., Int. J. Exp. Path. 92:400-412 (2011); and Rosa et al., Biomolecules 12, 184 (2022)
Biochemical measurements of markers such as cardiac PGF2, CGRP, PGP-9.5, GAP-43, Troponin T
Urodynamic and erectile physiology assessments carried out according to the protocols mentioned in Melman et al., BJU Int. 104(9):1292-300 (2009)

What is claimed is:

1. A method of treating diabetes-associated autonomic neuropathy in a subject in need thereof, the method comprising to the subject a therapeutically effective dose of exogenous interleukin-6 (IL-6).

2. The method of claim 1, wherein the autonomic neuropathy is associated with Type 1 diabetes.

3. The method of claim 1, wherein the autonomic neuropathy is associated with Type 2 diabetes.

4. The method of claim 1, wherein the IL-6 reduces a stomach or intestine symptom associated with autonomic neuropathy selected from the following: constipation, diarrhea, early satiety, nausea after eating, bowel movement control, swallowing, swollen abdomen, vomiting of undigested food, or a stomach or intestine symptom that is associated with related autonomic nerve degeneration.

5. The method of claim 1, wherein the IL-6 reduces a heart or lung symptom associated with autonomic neuropathy selected from the following: abnormal heart rate or rhythm, blood pressure drop with position, high blood pressure, or shortness of breath with activity or exercise.

6. The method of claim 1, wherein the IL-6 reduces a bladder symptom associated with autonomic neuropathy selected from the following: difficulty in initiation of urination, a feeling of incomplete bladder emptying, or uncontrolled urination.

7. The method of claim 1, wherein the IL-6 reduces one or more of the following symptoms associated with autonomic neuropathy: excessive or reduced sweating, heat intolerance associated with activity or exercise, erectile dysfunction, vaginal dryness, female orgasm difficulty, reduced pupil size, or unexpected weight loss.

8. The method of claim 1, wherein the IL-6 restores a physiological balance of parasympathetic/sympathetic tone in the subject.

9. The method of claim 8, wherein the IL-6 reduces tachycardia or bradycardia in the subject.

10. The method of claim 1, wherein the IL-6 is administered to the subject at a dose of 0.01 μg/kg to 10 μg/kg.

11. The method of claim 10, wherein the IL-6 is administered to the subject at a dose of 0.1 μg/kg to 1 μg/kg.
